(19) United States
(12) Patent Application Publication
(10) Pub. No.: US 2026/0020688 A1
(43) Pub. Date: Jan. 22, 2026

(54) AIR MATTRESS AND AIR PRESSURE CONTROL SYSTEM FOR REDUCING SNORING AND SLEEP APNEA

(71) Applicant: Dazhai HUANG, Irvine, CA (US)

(72) Inventor: Dazhai HUANG, Irvine, CA (US)

(21) Appl. No.: 18/774,774

(22) Filed: Jul. 16, 2024

(51) Int. Cl. A47C 27/08; A47C 27/10; A61F 5/56

(52) U.S. Cl. CPC A47C 27/083; A47C 27/082; A47C 27/10; A61F 5/56

(57) ABSTRACT

An air mattress and related air pressure control system, where the air mattress can change shapes to help reducing snoring and sleep apnea. The air mattress has individually inflatable and deflatable air compartments that can be inflated to different degrees to create a desired profile, such as a sloped profile, of the air mattress. When the air mattress is used for sleeping, a controller controls the inflation and deflation of the air compartments upon detecting a snoring sound. The air compartments are inflated by compressed air from a compressed air cylinder, rather than a pump, so as to reduce noise during operation. When the air mattress is not used for sleeping, the compressed air cylinder may be re-filled by a pump.

### BACKGROUND OF THE INVENTION

This invention relates to an air mattress and related air pressure control system, and in particular, it relates to an air mattress with controllable shapes for reducing snoring and sleep apnea.

Snoring and sleep apnea can lead to various health problems. The onset and severity of snoring are often related to the sleeping position; for example, people tend to snore less when sleeping on their sides as compared to on their backs.

Air mattresses designed to reduce snoring by changing the users' sleeping position have been disclosed. For example, U.S. Pat. No. 8,832,887 describes “A bedding product [which] comprises a mattress and padding and an inflatable bladder assembly. The bedding product may have a plurality of inflatable members located underneath select portions of the padding. The bedding product may have a sensor adapted to detect a sound associated with human snoring and an air supply system operatively coupled to the sensor and adapted to control a supply of air to one of the inflatable members.” (Abstract.) FIGS. 5A-5E of this patent show bladders being selectively inflated to change the shape of the padding to cause the user to turn to their sides.

As another example, U.S. Pat. No. 7,513,003 describes “A mattress supports a left-side user and a right-side user. At least one acoustic sensor is configured to sense acoustic energy in the environment of the left-side user and of the right-side user. At least one pressure sensor is configured to sense pressure applied to the left-side portion by the left-side user and applied to the right-side portion by the right side user. A controller is configured to receive at least one acoustic stream from the at least one acoustic sensor and at least one pressure stream from the at least one pressure sensor. Left-side snore/breath parameters and right-side snore/breath parameters are generated. The controller is further configured to, responsive to determining that a home automation rule includes a condition that includes at least one of the left-side snore/breath parameters and the right-side snore/breath parameters send an instruction to drive a controllable device to the controllable device.” (Abstract.)

As another example, U.S. Pat. No. 9,370,457 describes an air mattress system and associated “method . . . that includes measuring sound waves using a sound measuring device, determining, at a central controller of an air mattress system, one or more parameter values of the sound waves, comparing the one or more parameter values with values, ranges, or patterns indicative of snoring, identifying a snoring state of a user, and initiating, with the central controller, a change to one or more adjustable features of the air mattress system.” (Abstract.)

### SUMMARY OF THE INVENTION

The present invention is directed to an air mattress system and related method that substantially obviates one or more of the problems due to limitations and disadvantages of the related art.

An object of the present invention is to provide an air mattress system with an improved air pressure control system that reduces disruption of the user's sleep and improves sleep quality.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides an air mattress system, which includes: an air mattress including a plurality of independently inflatable and deflatable air compartments; a compressed air container configured to store compressed air; a controllable conduit and valve system, including a plurality of conduits coupled between the plurality of air compartments and the compressed air container and a plurality of valves on the plurality of conduits; and a controller coupled to the controllable conduit and valve system to independently control inflation of each air compartment by the compressed air container and/or deflation of each air compartment.

In another aspect, the present invention provides a method of operating an air mattress system, the air mattress system comprising an air mattress including a plurality of independently inflatable and deflatable air compartments, a compressed air container, and a controllable conduit and valve system coupled between the plurality of air compartments and the compressed air container, the method including: while the air mattress system is used for sleeping by a user: by an audio sensor, detecting a snoring sound of the user; and by a controller, in response to detecting the snoring sound of the user, controlling the controllable conduit and valve system to independently inflate some or all of the plurality of air compartments by compressed air stored in the compressed air container, and/or to independently deflate some or all of the plurality of air compartments, to form a sloped profile of the air mattress. The method may further include, while the air mattress system is not used for sleeping by the user, re-filling the compressed air container with compressed air by a pump.

It is to be understood that both the foregoing general description and the following detailed description are exemplary and explanatory and are intended to provide further explanation of the invention as claimed.

### BRIEF DESCRIPTION OF DRAWINGS

FIG. 1 schematically illustrates an air mattress and associated air pressure control system according to an embodiment of the present invention.

FIG. 2 schematically illustrates a cross-sectional view of the air mattress and associated components according to an embodiment of the present invention.

### DETAILED DESCRIPTION OF THE INVENTION

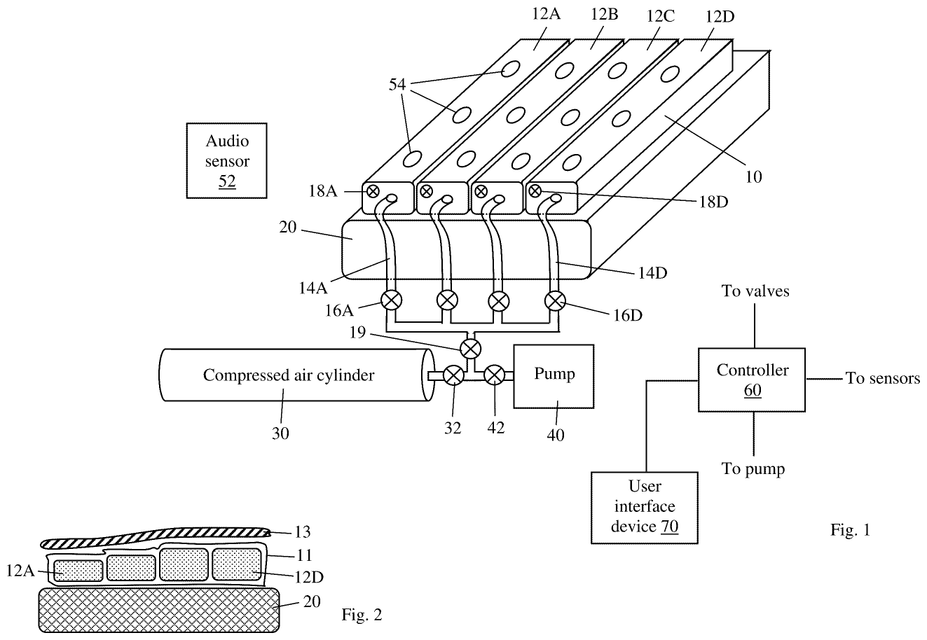

A segmented air mattress and associated air pressure control system are described with reference to FIGS. 1 and 2. The segmented air mattress may be inflated to different shapes, e.g., a gentle left-right sloped shape, to gently cause the user who is sleeping on the mattress to turn to sleep on their sides if they start to snore. The air pressure control system is designed to change the profile of the air mattress in a manner that minimizes disruption to the user's sleep.

The air mattress 10, which is suitable to be used on top of a bed 20, includes multiple separated air compartments (four in the illustrated embodiment) 12A-12D that are isolated from each other and can be independently inflated and deflated. In a preferred embodiment, the air compartments 12A-D are elongated in the longitudinal direction of the bed, and are arranged side-by-side across the transverse direction. The minimum number of air compartments is two, e.g., one on the left-hand side of the bed and one on the right-hand side of the bed. There may be odd or even numbers of air compartments. In alternative embodiments (not shown), each of the elongated air compartment may be replaced by a row of smaller independently inflatable air compartments arranged in the longitudinal direction.

The air compartments 12A-D are preferably enclosed in and held together by a flexible enclosure 11, or otherwise joined together by a flexible structure (e.g., sewn fabric, hook-and-loop fastening structure, etc.), to form an integral air mattress 10. A separate soft pad 13 may be placed over the air mattress 10 to enhance the comfort of the bed. The enclosure 11 or joining structure and the optional pad 13 are not shown in FIG. 1 to avoid obscuring the air compartments.

The segmented air mattress 10 is controlled to change its height profile to help the user sleep better. For example, as shown in FIG. 2, the air compartments 12A-D on the left and right-hand sides may be inflated to different degrees to create a gentle left-right slope of the air mattress 10 to gently cause the user to turn to their left or right side, preferably without waking them up. It is generally known that changing sleeping position may help stop snoring.

Preferably, the profile change of the air mattress 10 is activated only if snoring is detected. To this end, an audio sensor 52 is provided adjacent to the bed to sense the snoring sound of the user. When the user is not snoring, the air mattress is evenly inflated or not inflated and does not create a slope. When snoring is detected, the multiple air compartments are inflated or deflated to different degrees to create the left-right slope.

Optionally, pressure sensors 54 may be provided at multiple points over the mattress 10 to detect where on the mattress the user is located and to detect the user's sleeping position (e.g., on their back or on their side). FIG. 1 shows pressure sensors 54 located on top of the air compartments 12A-D, but they may alternatively be located at the bottom of the air compartments, or located on the separate pad 13 placed over the air compartments. The information sensed by the pressure sensors 54 may be used to control the inflation and deflation of the air compartments (once the sound sensor detects snoring), e.g., whether the air mattress should be sloped down toward the left or the right, which air compartment should be inflate/deflated to what degree, etc. For an air mattress with a more complex designs of air compartments (e.g., the number of rows, the number of longitudinally arranged compartments in each row, etc.), more complex algorithms to control optimum inflation profiles of the air mattress is provided.

According to embodiments of the present invention, the inflation of the air compartments 12A-D is accomplished using compressed air from a compressed air container (cylinder) 30. In other words, air pressure is not directly generated by an air pump (which can be noisy) when the user is using the air mattress to sleep. Preferably, the inflating and deflating process is relatively slow, e.g., over the course of a minute or longer after snoring is detected. This can reduce the hissing noise generated when the air flows through the valves. This relatively slow inflation process also reduces the chance of waking up the user by sudden changes. The compressed air container 30 may be re-filled by an air pump 40 when the air mattress is not used for sleeping.

More specifically, each air compartment 12A etc. is connected to the compressed air container 30 by a conduit 14A etc. with an air inlet valve 16A etc. Each air compartment 12A etc. may have an air release valve 18A etc. separate from the air inlet valve 16A etc.; alternatively, each air inlet valve 16A etc. may be a three-way valve that may be turned to a position to release air from the air compartment, so a separate air release valve is not needed. Optionally, a master valve 19 between the compressed air container 30 and the individual air inlet valves 16A etc. may be provided. The air inlet valves 16A etc. for the individual air compartments, the air release valves 18A etc. (if separate from the air inlet valves), and the optional master valve 19, along with the conduits 14A etc., collectively form a controllable conduit and valve system between the compressed air container 30 and the air compartments 12A etc. that allows each air compartment to be inflated and deflated to desired degrees.

The compressed air container 30 has a cylinder valve (regulator) 32, and the pump 40 has a pump valve (regulator) 42. When the air mattress 10 is used for sleeping, the pump 40 is turned off and the pump valve 42 is closed. The cylinder valve 32 and the optional master valve 19 are open, and the air inlet valves 16A etc. and the air release valves 18A etc. are controlled to open or close, so that the compressed air from the container 30 selectively inflates the air compartments to change the profile of the air mattress 10. When the air mattress 10 is not used for sleeping, the compressed air container 30 may be re-filled by the pump 40, by closing the mattress master valve 19 (or the individual valves 16A etc. if a master valve is not present), opening the cylinder valve 32 and the pump valve 42, and turning on the pump 40 to produce compressed air. Both the compressed air container 30 and the pump 40 are preferably located near the bed 20, and the compressed air container 30 does not need to be moved or removed in order for it to be re-filled.

The various valves described above may have any suitable structure, which are generally known in the art. They may be simple on-off valves or pressure regulators. Each valve has an electrical component that drives the opening and closing of the valve. The operations of the valves and other components are controlled by a controller 60, which is electrically and/or communicatively coupled (by wired and/or wireless coupling) to the valves 16A etc., 18A etc., 19, 32 and 42, the audio sensor 52 and pressure sensors 54, and the pump 40. Based on the signals sensed by the audio sensor and/or the pressure sensors, the controller 60 controls the opening and closing of the various valves to inflate and deflate the individual air compartments to create desired profiles of the mattress to reduce snoring.

The controller 60 is preferably also coupled to a user interface device 70, such as a control panel (e.g., a panel mounted on the side of the bed or on the wall of the bedroom), a handheld device (e.g., a remote control device, a smart phone, etc.), etc., which allows the user to interact with the controller. The controller 60 may include a microcontroller or any other suitable electronic circuits.

It will be apparent to those skilled in the art that various modification and variations can be made in the air mattress and related operation method of the present invention without departing from the spirit or scope of the invention. Thus, it is intended that the present invention cover modifications and variations that come within the scope of the appended claims and their equivalents.

1. An air mattress system comprising:
an air mattress including a plurality of independently inflatable and deflatable air compartments;
a compressed air container configured to store compressed air;
a controllable conduit and valve system, including a plurality of conduits coupled between the plurality of air compartments and the compressed air container and a plurality of valves on the plurality of conduits; and
a controller coupled to the controllable conduit and valve system to independently control inflation of each air compartment by the compressed air container and/or deflation of each air compartment.

2. The air mattress system of claim 1, wherein the controllable conduit and valve system further includes a plurality of air release valves respectively on the plurality of air compartments.

3. The air mattress system of claim 1, wherein the plurality of air compartments are elongated in the longitudinal direction of the air mattress and are arranged side-by-side across a transverse direction.

4. The air mattress system of claim 1, further comprising an audio sensor coupled to the controller, configure to detect a snoring sound of a user;
wherein the controller is configured to, in response to the audio sensor detecting the snoring sound, control the controllable conduit and valve system to individually inflate and/or deflate some or all of the plurality of air compartments to form a sloped profile of the air mattress.

5. The air mattress system of claim 4, further comprising a plurality of pressure sensors coupled to the controller, the pressure sensors being located above and/or below the air mattress and configure to detect a location and/or a sleeping position of the user;
wherein the controller is configured to, based on the location and/or sleeping position of the user detected by the pressure sensors, control the controllable conduit and valve system to individually inflate and/or deflate some or all of the plurality of air compartments to form the sloped profile of the air mattress.

6. The air mattress system of claim 4, wherein the controller is configured to control the controllable conduit and valve system to form the sloped profile of the air mattress over a time interval of a minute or longer after the audio sensor detects the snoring sound.

7. A method of operating an air mattress system, the air mattress system comprising an air mattress including a plurality of independently inflatable and deflatable air compartments, a compressed air container, and a controllable conduit and valve system coupled between the plurality of air compartments and the compressed air container, the method comprising:
while the air mattress system is used for sleeping by a user:
by an audio sensor, detecting a snoring sound of the user; and
by a controller, in response to detecting the snoring sound of the user, controlling the controllable conduit and valve system to independently inflate some or all of the plurality of air compartments by compressed air stored in the compressed air container, and/or to independently deflate some or all of the plurality of air compartments, to form a sloped profile of the air mattress.

8. The method of operating an air mattress system of claim 7, further comprising:
while the air mattress system is not used for sleeping by the user, re-filling the compressed air container with compressed air by a pump.

9. The method of operating an air mattress system of claim 7, further comprising:
while the air mattress system is used for sleeping by the user:
by a plurality of pressure sensors located above and/or below the air mattress, detecting a location and/or a sleeping position of the user; and
based on the location and/or sleeping position of the user detected by the pressure sensors, controlling the controllable conduit and valve system to individually inflate and/or deflate some or all of the plurality of air compartments to form the sloped profile of the air mattress.

10. The method of operating an air mattress system of claim 7, wherein the controller is configured to control the controllable conduit and valve system to form the sloped profile of the air mattress over a time interval of a minute or longer after the audio sensor detects the snoring sound.
